(19) United States
(12) Patent Application Publication
(10) Pub. No.: US 2005/0100987 A1
(43) Pub. Date: May 12, 2005

(54) Intein-mediated protein ligation of expressed proteins

(76) Inventors: Thomas C. Evans, Somerville, MA (US); Ming qun Xu, Hamilton, MA (US); Shaorong S.C. Chong, Somerville, MA (US)

Correspondence Address: Harriet M. Strimpel;New England Biolabs, Inc., 32 Tozer Road, Beverly, MA, 01915, US

(21) Appl. No.: 10/960,905

(22) Filed: Oct. 7, 2004

Related U.S. Application Data
(63) Continuation of application No. 09/249,543, filed on Feb. 12, 1999, now Pat. No. 6,849,428.
(60) Provisional application No. 60/102,413, filed on Sep. 30, 1998.

(51) Int. Cl. C12P 21/06; C07H 21/04; C12N 9/00; C12N 1/18; C12N 5/06; C12N 15/74

(52) U.S. Cl. 435/69.1; 435/320.1; 435/471; 435/325; 435/348; 435/254.2; 435/252.1; 435/183; 530/350; 536/23.2

(57) ABSTRACT

A method for the ligation of expressed proteins which utilizes inteins, for example the RIR1 intein from Methanobacterium thermotrophicum , is provided. Constructs of the Mth RIR1 intein in which either the C-terminal asparagine or N-terminal cysteine of the intein are replaced with alanine enable the facile isolation of a protein with a specified N-terminal, for example, cysteine for use in the fusion of two or more expressed proteins. The method involves the steps of generating a C-terminal thioester-tagged target protein and a second target protein having a specified N-terminal via inteins, such as the modified Mth RIR1 intein, and ligating these proteins. A similar method for producing a cyclic or polymerized protein is provided. Modified inteins engineered to cleave at their C-terminus or N-terminus, respectively, and DNA and plasmids encoding these modified inteins are also provided.

### RELATED APPLICATIONS

This Application is a Continuation application of U.S. application Ser. No. 09/249,543, filed Feb. 12, 1999, which claims priority from U.S. provisional application No. 60/102,413, filed Sep. 30, 1998.

### BACKGROUND OF THE INVENTION

The present invention relates to methods of intein-mediated ligation of proteins. More specifically, the present invention relates to intein-mediated ligation of expressed proteins containing a predetermined N-terminal residue and/or a C-terminal thioester generated via use of one or more naturally occurring or modified inteins. Preferably, the predetermined residue is cysteine.

Inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al.,EMBO J. 15(19):5146-5153 (1996)).

Studies into the mechanism of intein splicing led to the development of a protein purification system that utilized thiol-induced cleavage of the peptide bond at the N-terminus of the Sce VMA intein (Chong et al., Gene 192(2):271-281 (1997)). Purification with this intein-mediated system generates a bacterially-expressed protein with a C-terminal thioester (Chong et al., (1997)). In one application, where it is described to isolate a cytotoxic protein, the bacterially expressed protein with the C-terminal thioester is then fused to a chemically-synthesized peptide with an N-terminal cysteine using the chemistry described for “native chemical ligation” (Evans et al., Protein Sci. 7:2256-2264 (1998); Muir et al., Proc. Natl. Acad. Sci. USA 95:6705-6710 (1998)).

This technique, referred to as “intein-mediated protein ligation” (IPL), represents an important advance in protein semi-synthetic techniques. However, because chemically-synthesized peptides of larger than about 100 residues are difficult to obtain, the general application of IPL is limited by the requirement of a chemically-synthesized peptide as a ligation partner.

IPL technology would be significantly expanded if an expressed protein with a predetermined N-terminus, such as cysteine, could be generated. This would allow the fusion of one or more expressed proteins from a host cell, such as bacterial, yeast or mammalian cells.

One method of generating an N-terminal cysteine is with the use of proteases. However, proteases have many disadvantages, such as the possibility of multiple protease sites within a protein, as well as the chance of non-specific degradation. Furthermore, following proteolysis, the proteases must be inactivated or purified away from the protein of interest before proceeding with IPL. (Xu, et al., Proc. Natl. Acad. Sci. USA 96(2):388-393 (1999) and Erlandson, et al., Chem. Biol., 3:981-991 (1996))

There is, therefore, a need for an improved intein-mediated protein ligation method which overcomes the noted limitations of current IPL methods and which eliminates the need for use of proteases to generate an N-terminal cysteine residue. Such an improved IPL method would have widespread applicability for the ligation of expressed proteins, for example, labeling of extensive portions of a protein for, among other things, NMR analysis.

### SUMMARY OF THE INVENTION

In accordance with the present invention, there is provided a method for the ligation of expressed proteins utilizing one or more inteins which display cleavage at their N— and/or C-termini. In accordance with the present invention, such inteins may occur either naturally or may be modified to cleave at their N— and/or C-termini. Inteins displaying N— and/or C-terminal cleavage enable the facile isolation of a protein having a C-terminal thioester and a protein having an N-terminal amino acid residue such as cysteine, respectively, for use in the fusion of one or more expressed proteins. Alternatively, the method may be used to generate a single protein having both a C-terminal thioester and a specified N-terminal amino acid residue, such as cysteine, for the creation of cyclic or polymerized proteins. These methods involve the steps of generating at least one C-terminal thioester-tagged first target protein, generating at least one second target protein having a specified N-terminal amino acid residue, for example cysteine, and ligating these proteins. This method may be used where a single protein is expressed, where, for example, the C-terminal thioester end of the protein is fused to the N-terminal end of the same protein. The method may further include chitin-resin purification steps.

In one preferred embodiment the intein from the RIR1 Methanobacterium thermoautotrophicum is modified to cleave at either the C-terminus or N-terminus. The modified intein allows for the release of a bacterially expressed protein during a one-column purification, thus eliminating the need proteases entirely. DNA encoding these modified inteins and plasmids containing these modified inteins are also provided by the instant invention.

### BRIEF DESCRIPTION OF THE DRAWINGS

FIG. 1 is a diagram depicting both the N-terminal and C-terminal cleavage reactions which comprise intein-mediated protein ligation. The modified Mth RIR1 intein was used to purify both MBP with a C-terminal thioester and T4 DNA ligase with an N-terminal cysteine. The Mth RIR1 intein for N-terminal cleavage, intein(N), carried the P⁻¹G/N¹³⁴A double mutation. The full length fusion protein consisting of MBP-intein(N)-CBD was separated from cell extract by binding the CBD portion of the fusion protein to a chitin resin. Overnight incubation in the presence of 100 mM 2-mercaptoethanesulfonic acid (MESNA) induced cleavage of the peptide bond prior to the N-terminus of the intein and created a thioester on the C-terminus of MBP. The C-terminal cleavage vector, intein(C), had the P⁻¹G/C¹A double mutation. The precursor CBD-intein(C)-T4 DNA ligase was isolated from induced E. coli cell extract by binding to a chitin resin as described for N-terminal cleavage. Fission of the peptide bond following the C-terminal residue of the intein at a preferred temperature and pH resulted in the production of T4 DNA ligase with an N-terminal cysteine. Ligation occurred when the proteins containing the complementary reactive groups were mixed and concentrated, resulting in a native peptide bond between the two reacting species.

FIG. 2A is a gel depicting the purification of a C-terminal thioester-tagged maltose binding protein (MBP) via a thiol-inducible Mth RIR1 intein construct pMRB10 G (containing the modified intein, R(N), with P⁻¹G/N¹³⁴A mutation) and the purification of T4 DNA ligase having an N-terminal cysteine using the vector PBRL-A (containing the modified intein, R(C), with P⁻¹G/C¹A mutation). Lanes 1-3, purification of maltose binding protein (MBP) (M, 43 kDa) with a C-terminal thioester. Lane 1. ER2566 cells transformed with plasmid pMRB10G following Isopropyl β-D-thiogalactopyranoside (IPTG) induction. Lane 2. Cell extract after passage over a chitin resin. Note that the fusion protein, M-R(N)—B, binds to the resin, where B is the chitin binding domain. Lane 3. Fraction 3 of the elution from the chitin resin following overnight incubation at 40° C. in the presence of 100 mM MESNA. Lanes 4-6, purification of T4 DNA ligase (L, 56 kDa) with an N-terminal cysteine. Lane 4. IPTG induced ER2566 cells containing plasmid pBRL-A. Lane 5. Cell extract after application to a chitin resin. B-R(C)-L, the fusion protein, binds to the resin. Lane 6. Elution of T4 DNA ligase with an N-terminal cysteine after overnight incubation at room temperature in pH 7 buffer

FIG. 2B is a gel depicting ligation of T4 DNA ligase having an N-terminal cysteine to a C-terminal thioester tagged MBP. Lane 1. Thioester-tagged MBP. Lane 2. T4 DNA ligase with an N-terminal cysteine. Lane 3. Ligation reaction of MBP (0.8 mM) with T4 DNA ligase (0.8 mM), generating M-L, after overnight incubation at 40° C.

FIG. 3 is a gel depicting the effect of induction temperature on the cleaving and/or splicing activity of the Mth RIR1 intein or Mth RIR1 intein mutants. The Mth RIR1 intein or mutants thereof, with 5 native N— and C-terminal extein residues were induced at either 15° C. or 37° C. The intein was expressed as a fusion protein (M-R—B, 63 kDa) consisting of N-terminal maltose binding protein (M, 43 kDa), the Mth RIR1 intein (R, 15 kDa) and at its C-terminus was the chitin binding domain (B, 5 kDa). Lanes 1 and 2. M-R—B with the unmodified Mth RIR1 intein. Note the small amount of spliced product (M-B, 48 kDa). Lanes 3 and 4. Mth intein with Pro^{31 ‘}replaced with Ala, M-R—B(P⁻¹A). Both spliced product (M-B) and N-terminal cleavage product (M) are visible. Lanes 5 and 6. Replacement of Pro⁻¹ with Gly (M-R—B (P⁻¹G)) showed some splicing as well as N— and C-terminal cleavage, M and M-R, respectively. Lanes 7 and 8. The Pro⁻to Gly and Cys¹ to Ser double mutant, M-R—B(P⁻¹G/C¹S), displayed induction temperature dependent C-terminal cleavage (M-R) activity. Lanes 9 and 10. The M-R—B(P⁻¹G/N¹³⁴A) mutant possessed only N-terminal cleavage activity producing M. The Mth intein or Mth intein-CBD fusion is not visible in this Figure.

FIG. 4 is a nucleotide sequence (SEQ ID NO:23) comparison of wild type Mth RIR1 intein and synthetic Mth RIR1 intein indicating the location of 61 silent base mutations designed to increase expression in E. coli. DNA alignment of the wild type Mth RIR1 intein (top strand) (SEQ ID NO:23) and the synthetic Mth RIR1 intein (bottom strand) (SEQ ID NO:25). To increase expression levels in E. coli, 61 silent base changes were made in 49 separate codons when creating the synthetic gene. The first and last codons of the wild type Mth RIR1 intein are shown in bold.

### DETAILED DESCRIPTION

The present invention provides a solution to the limitations of current intein-mediated ligation methods by eliminating the need for a synthetic peptide as a ligation partner, and providing a method which is suitable for the fusion one or more expressed proteins.

In general, any intein displaying N— and/or C-terminal cleavage at its splice junctions can be used to generate a defined N-terminus, such as cysteine as well as a C-terminal thioester for use in the fusion of expressed proteins. Inteins which may be used in practicing the present invention include those described in Perler, et al., Nucleic Acids Res., 27(1):346-347 (1999).

In accordance with one preferred embodiment, an intein found in the ribonucleoside diphosphate reductase gene of Methanobacterium thermoautotrophicum (the Mth RIR1 intein) was modified for the facile isolation of a protein with an N-terminal cysteine for use in the in vitro fusion of two bacterially-expressed proteins. The 134-amino acid Mth RIR1 intein is the smallest of the known mini-inteins, and may be close to the minimum amino acid sequence needed to promote splicing (Smith et.al., J. Bacteriol. 179:7135-7155 (1997)).

The Mth RIR1 intein has a proline residue on the N-terminal side of the first amino acid of the intein. This residue was previously shown to inhibit splicing in the Sce VMA intein (Chong et al., J. Biol. Chem. 273:10567-10577 (1998)). The intein was found to splice poorly in E. coli when this naturally occurring proline is present. Splicing proficiency increases when this proline is replaced with an alanine residue. Constructs that display efficient N— and C-terminal cleavage are created by replacing either the C-terminal asparagine or N-terminal cysteine of the intein, respectively, with alanine.

These constructs allow for the formation of an intein-generated generated C-terminal thioester on a first target protein and an intein-generated N-terminal cysteine on a second target protein. These complementary reactive groups may then be ligated via native chemical ligation to produce a peptide bond (Evans et al supra (1998), Muir et al supra (1998)). Alternatively, a single protein containing both reactive groups may be generated for the creation of cyclic or polymerized proteins. Likewise, more than one first or second target proteins may be generated via use of multiple mutant inteins.

As used herein, the terms fusion and ligation are used interchangeably. Also as used herein, protein shall mean any protein, fragment of any protein, or peptide capable of ligation according to the methods of the instant invention. Further, as used herein, target protein shall mean any protein the ligation of which, according to the methods of the instant invention, is desired.

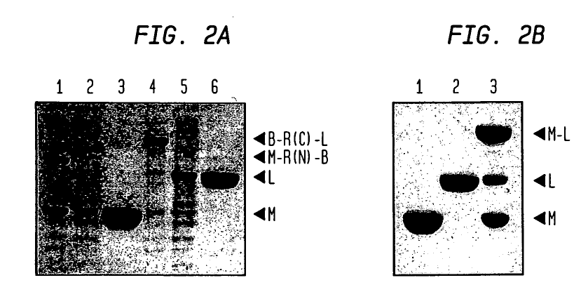

The general method of intein-mediated protein ligation in accordance with the present invention is as follows:
(1) An intein of interest is isolated and cloned into an appropriate expression vector(s) such as bacterial, plant, insect, yeast and mammalian cells.
(2) The intein is engineered for N— and/or C-terminal cleavage unless the wild type intein displays the desired cleavage activities. In a preferred embodiment, a modified intein with the desired cleavage properties can be generated by substituting one or more residues within and/or flanking the intein sequence. For example, a modified intein having N-terminal cleavage activity can be created by changing the last intein residue. Alternatively, a modified intein with C-terminal cleavage activity can be created by changing the first intein residue.
(3) The intein with N— and/or C-terminal cleavage activity is fused with an affinity tag to allow purification away from other endogenous proteins.
(4) The intein or inteins, either wild type or modified, that display N-terminal and/or C-terminal cleavage, or both, are fused to the desired target protein coding region or regions upstream and/or downstream of the intein.
(5) An intein that cleaves at its N-terminus in a thiol reagent dependent manner is used to isolate a protein with a C-terminal thioester. This cleavage and isolation is, for example, carried out as previously described for the Sce VMA and Mxe GyrA inteins (Chong et al., Gene 192(2):271-281 (1997); Evans et al., Protein Sci. 7:2256-2264 (1998)). As discussed previously, multiple C-terminal thioester-tagged proteins may be generated at this step .
(6) A target protein having a specified N-terminus is generated by cleavage of a construct containing an intein that cleaves at its C-terminus. The specified N-terminal residue may be any of the amino acids, but preferably cysteine. As discussed previously, this step may alternately generate a specified N-terminal on the same protein containing a C-terminal thioester, to yield a single protein containing both reactive groups. Alternatively, multiple proteins having the specified N-terminus may be generated at this step.
(7) Thioester-tagged target protein and target protein having a specified N-termini are fused via intein-mediated protein ligation (IPL) (see FIG. 2B). In a preferred embodiment, the N-terminus is cysteine. Alternatively, a single protein containing both a C-terminal thioester and a specified N-terminus, such as a cysteine, may undergo intramolecular ligation to yield a cyclic product and/or intermolecular ligation to yield polymerized proteins.

The methodology described by the instant invention significantly expands the utility of current IPL methods to enable the labeling of extensive portions of a protein for NMR analysis and the isolation of a greater variety of cytotoxic proteins. In addition, this advance opens the possibility of labeling the central portion of a protein by ligating three or more fragments.

The use of an intein or inteins with N-terminal and C-terminal cleavage activity provides the potential to create a defined N-terminus, such as a cysteine, and a C-terminal thioester on a single protein. The intramolecular ligation of the resulting protein generates a circular protein, whereas the intermolecular ligation of several of these proteins generates a protein polymer.

Cleavage at the N— and/or the C-terminus of an intein can be brought about by introducing changes to the intein and/or its extein sequences. Also, naturally occuring inteins may display these properties and require no manipulation. Cleavage at the N— and/or C-terminus of an intein can occur uncontrollably or induced using nucleophilc compounds, such as thiol reagents, temperature, pH, salt, chaotropic agents, or any combination of the aforementioned conditions and/or reagents.

The Examples presented below are only intended as specific preferred embodiments of the present invention and are not intended to limit the scope of the invention except as provided in the claims herein. The present invention encompasses modifications and variations of the methods taught herein which would be obvious to one of ordinary skill in the art.

The references cited above and below are herein incorporated by reference.

### EXAMPLE I

### Creation of the Mth RIR1 Synthetic Gene

The gene encoding the Mth RIR1 intein along with 5 native N— and C-extein residues (Smith et al. supra (1997)) was constructed using 10 oligonucleotides (New England Biolabs, Beverly, Mass.) comprising both strands of the gene, as follows:

(SEQ ID NO:1)

| | |
|---|---|
| 1) | 5′-TGGAGGCAACCAACCCCTGCGTATCGGGTGACACCATTGT |
| | AATGACTAGTGGCGGTGCGCGCACTGTGGGTGAACTGGAGGGG |
| | AAACCGTTCACCGCAC-3′ |

(SEQ ID NO:2)

| | |
|---|---|
| 2) | 5′-GGGGTTGGGTGCTGGCCACAGTTGTGTACAATGAAGGCAT |
| | TAGCAGTGAATGCGCTAGCACCGTAAACAGTAGCGTGATAAAC |
| | ATGCTGGCGG-3′ |

(SEQ ID NO:3)

| | |
|---|---|
| 3) | 5′-pTGATTCGCGGCTGTGGCTAGCGATGGGGCTCAGGTTTCT |
| | TCCGCACCTGTGAACGTGACGTATATGATCTGCGTAGACGTGA |
| | GGGTCATTGCTTAGGTTT-3′ |

(SEQ ID NO:4)

| | |
|---|---|
| 4) | 5′-pGACGCATGATGACCGTGTTCTGGTGATGGATGGTGGCCT |
| | GGAATGGGGTGCCGGGGGTGAACTGGAACGCGGGGACCGGCTG |
| | GTGATGGATGATGCAGCT-3′ |

(SEQ ID NO:5)

| | |
|---|---|
| 5) | 5′-pGGCGAGTTTGCGGCACTGGGAACCTTGCGTGGCGTGCGT |
| | GGGGCTGGGGGGGAGGATGTTTATGAGGGTACTGTTTAcGGTG |
| | GTAGC-3′ |

(SEQ ID NO:6)

| | |
|---|---|
| 6) | 5′-pGGATTGAGTGGTAATGGGTTGATTGTACAGAACTGTGGC |
| | GAGCAGCGAA-3′ |

(SEQ ID NO:7)

| | |
|---|---|
| 7) | 5′-pCCAGGGGGACGCAGGCCACGGAAGGTTGCCAGTGCCGGA |
| | AACTCGCCAGGTGGATGATGGATGACGAGGCGGTGGGGGCGTT |
| | CGAGTTCACCCGCGGCAC-3′ |

(SEQ ID NO:8)

| | |
|---|---|
| 8) | 5′-pGCCATTCCAGGCCACCATCCATGAGCAGAACAGGGTGAT |
| | CATGGGTCAAAGGTAAGCAATGAGCGTCACGTGTACGGAGATC |
| | ATATAGGT-3′ |

(SEQ ID NO:9)

| | |
|---|---|
| 9) | 5′-pCAGGTTGACAGGTGCGGAAGAAAGGTGAGGGGCATGGGT |
| | AGCGAGAGGCGCGAATGAGTGGGGTGAAGGGTTTGGGGTGCAG |
| | TTCAGCCAGAGTGCG-3′ |

(SEQ ID NO:10)

| | |
|---|---|
| 10) | 5′-pCGGAGGGCCAGTAGTGATTAGAATGGTGTCAGGGGATAC |
| | GCAGGGGTTGGTTGCC-3′ |

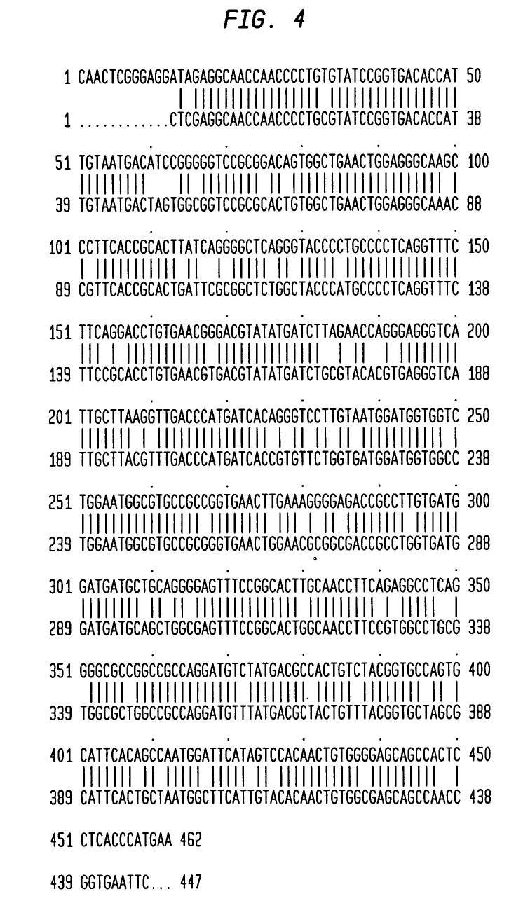

To ensure maximal E. coli expression, the coding region of the synthetic Mth RIR1 intein incorporates 61 silent base mutations in 49 of the 134 codons (see FIG. 4) in the wildtype Mth 261 intein gene (GenBank AE000845). The oligonucleotides were annealed by mixing at equimolar ratios (400 nM) in a ligation buffer (50 mM Tris-HCI, pH 7.5 containing 10 mM MgCl₂, 10 mM dithiothreitol, 1 mM ATP, and 25 μg BSA) followed by heating to 95° C. After cooling to room temperature, the annealed and ligated oligonucleotides were inserted into the Xhol and Agel sites of pMYB5 (NEB), replacing the Sce VMA intein and creating the plasmid pMRB8P.

### Engineering the Mth 261 Intein for N— and C-terminal Cleavage

The unique Xhol and Spel sites flanking the N-terminal splice junction and the unique BsrGl and Agel sites flanking the C-terminal splice junction allowed substitution of amino acid residues by linker replacement. The proline residue, Pro⁻¹, preceding the intein in pMRB8P was substituted with alanine or glycine to yield pMRB8A and pMRB8G1, respectively. Substitution of Pro⁻¹-Cys¹ with Gly-Ser or Gly-Ala yielded pMRB9GS and pMRB9GA, respectively. Replacing Asn¹³⁴ with Ala in pMRB8G1 resulted in pMRB1 OG. The following linkers were used for substitution of the native amino acids at the splice junctions (each linker was formed by annealing two synthetic oligonucleotides as described above):

| | |
|---|---|
| P- ¹A linker: | |
| 5′-TCGAGGCAACGAAGGGATGGGTATCCGGT | (SEQ ID NO:11) |
| GACAGGATTGTAATGA-3′ | |
| and | |
| 5 ′-GTAGTGATTAGAATGGTGTGACGGGATAC | (SEQ ID NO:12) |
| GCATGCGTTGGTTGGG-3′ | |
| P- ¹G linker: | |
| 5′-TGGAGGGCTGCGTATCCGGTGAGAGGATT | (SEQ ID NO:13) |
| GTAATGA-3′ | |
| and | |
| 5′-CTAGTGATTAGAATGGTGTCACGGGATAC | (SEQ ID NO:14) |
| GGAGCGG-3′ | |
| P- ¹G/C¹S linker: | |
| 5′-TGGAGGGCATCGAGGGAAGGAAGGGATC | (SEQ ID NO:15) |
| CGTATCGGGTGAGAGGATTGTAATGA-3′ | |
| and | |
| 5′-GTAGTCATTACAATGGTGTCACCGGATAC | (SEQ ID NO:16) |
| GGATCCGTTGGTTGCCTGGATGCCC-3′ | |
| P- ¹G/C¹A linker: | |
| 5′-TCGAGGGGATCGAGGCAAGCAACGGCGCC | (SEQ ID NO:17) |
| GTATCCGGTGACACCATTGTAATGA-3′ | |
| and | |
| 5 ′-CTAGTCATTAGAATGGTGTCACCGGATAC | (SEQ ID NO:18) |
| GGGGCGGTTGGTTGGGTGGATGCGC-3′ | |
| N¹³⁴A linker: | |
| 5′-GTAGACGCATGCGGCGAGGAGCCCGG | (SEQ ID NO:19) |
| GA-3′ | |
| and | |
| 5′-CGGGTCCCGGGCTGGTCGCGGCATGC | (SEQ ID NO:20) |
| GT-3′ | |

pBRL-A was constructed by substituting the Escherichia coli maltose binding protein (MBP) and the Bacillus circulans chitin binding domain (CBD) coding regions in pMRB9GA with the CBD and the T4 DNA ligase coding regions, respectively, subcloned from the pBYT4 plasmid.

### EXAMPLE II

### Generating a Thioester-Tagged Protein

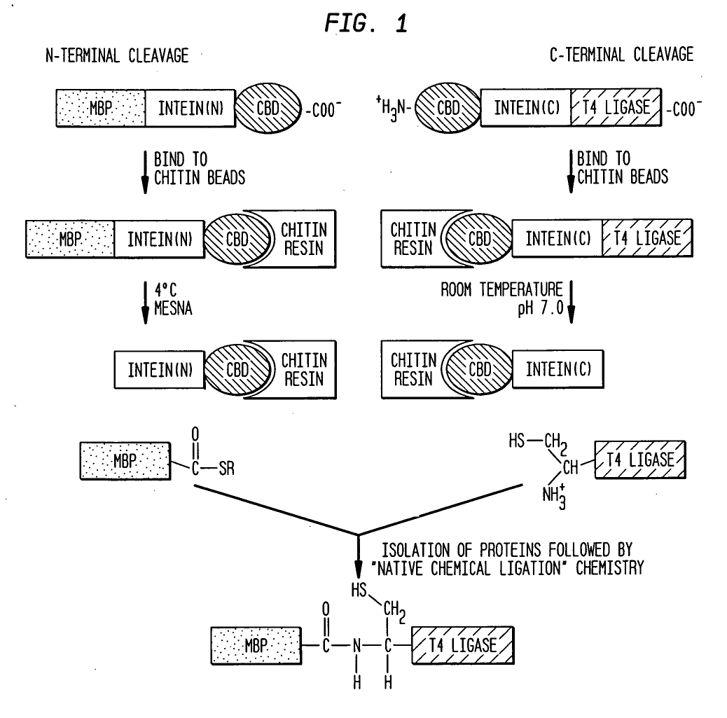

The pMRB10G construct from Example I contains the Mth RIR1 intein engineered to undergo thiol reagent induced cleavage at the N-terminal splice junction (FIG. 1, N-terminal cleavage) and was used to isolate proteins with a C-terminal thioester as described previously for the Sce VMA and Mxe GyrA inteins (Chong et al. supra 1997); Evans et al., supra (1998)). Briefly, ER2566 cells (Evans et.al. (1998)) containing the appropriate plasmid were grown at 37° C. in LB broth containing 100 μg/mL ampicillin to an OD₆₀₀ of 0.5-0.6 followed by induction with IPTG (0.5 mM). Induction was either overnight at 15° C. or for 3 hours at 30° C.

The cells were pelleted by centrifugation at 3,000×g for 30 minutes followed by resuspension in buffer A (20 mM Tris-HCl, pH 7.5 containing 500 mM NaCI). The cell contents were released by sonication. Cell debris was removed by centrifugation at 23,000×g for 30 minutes and the supernatant was applied to a column packed with chitin resin (10 mL bed volume) equilibrated in buffer A. Unbound protein was washed from the column with 10 column volumes of buffer A.

Thiol reagent-induced cleavage was initiated by rapidly equilibrating the chitin resin in buffer B (20 mM Tris-HCI, pH 8 containing 500 mM NaCI and 100 mM 2-mercaptoethane-sulfonic acid (MESNA)). The cleavage reaction, which simultaneously generates a C-terminal thioester on the target protein, proceeded overnight at 4° C. after which the protein was eluted from the column. The use of the PMRB10G construct resulted in the isolation of MBP with a C-terminal thioester (FIG. 2A).

### Isolating Proteins With An N-Terminal Cysteine

The pBRL-A construct from Example I contains an Mth RIR1 intein engineered to undergo controllable cleavage at its C-terminus, and was used to purify proteins with an N-terminal cysteine (FIG. 1, C-terminal cleavage). The expression and purification protocol was performed as described in Example II, except with buffer A replaced by buffer C (20 mM Tris-HCI, pH 8.5 containing 500 mM NaCI) and buffer B replaced by buffer D (20 mM Tris-HCI, pH 7.0 containing 500 mM NaCI). Also, following equilibration of the column in buffer D the cleavage reaction proceeded overnight at room temperature.

The expression of plasmid pBRL-A resulted in the purification of 4-6 mg/L cell culture of T4 DNA ligase possessing an N-terminal cysteine (FIG. 2A). Protein concentrations were determined using the Bio-Rad protein assay (Bio-Rad Laboratories, Inc., Hercules, Calif).

### EXAMPLE III

### Protein-Protein Ligation Using Intein-Mediated Protein Ligation

Intein-mediated protein ligation (IPL) was used to fuse two proteins (FIG. 2B). Freshly isolated thioester-tagged protein from Example II was mixed with freshly isolated protein containing an N-terminal cysteine residue from Example II, with typical starting concentrations of 1-200 μM. The solution was concentrated with a Centriprep 3 or Centriprep 30 apparatus (Millipore Corporation, Bedford, Mass.) then with a Centricon 3 or Centricon 10 apparatus to a final concentration of 0.15-1.2 mM for each protein.

Ligation reactions proceeded overnight at 40° C. and were visualized using SDS-PAGE with 12% Tris-glycine gels (Novex Experimental Technology, San Diego, Calif.) stained with Coomassie Brilliant Blue. Typical ligation efficiencies ranged from 20-60%.

### Confirmation Of Ligation In IPL Reactions

A Factor Xa site in MBP that exists 5 amino acids N-terminal terminal from the site of fusion (Maina et al, supra (1988)) allowed amino acid sequencing through the ligation junction. The sequence obtained was NH₂-TLEGCGEQPTGXLK—COOH (SEQ ID NO:21 ) which matched the last 4 residues of MBP (TLEG) followed by a linker sequence (CGEQPTG (SEQ ID NO:22)) and the start of T4 DNA ligase (ILK). During amino acid sequencing, the cycle expected to yield an isoleucine did not have a strong enough signal to assign it to a specific residue, so it was represented as an X. The cysteine was identified as the acrylamide alkylation product.

The Factor Xa proteolysis was performed on 2 mg of ligation reaction involving MBP and T4 DNA ligase. This reaction mixture was bound to 3 mL of amylose resin (New England Biolabs, Inc., Beverly, Mass.) equilibrated in buffer A (see Example II). Unreacted T4 DNA ligase was rinsed from the column with 10 column volumes of buffer A. Unligated MBP and the MBP-T4 DNA ligase fusion protein were eluted from the amylose resin using buffer E (20 mM Tris-HCI, pH 07.5 containing 500 mM NaCI and 10 mM maltose). Overnight incubation of the eluted protein with a 200:1 protein:bovine Factor Xa (NEB) ratio (w/w) at 40° C. resulted in the proteolysis of the fusion protein and regeneration of a band on SDS-PAGE gels that ran at a molecular weight similar to T4 DNA ligase. N-terminal amino acid sequencing of the proteolyzed fusion protein was performed on a Procise 494 protein sequencer (PE Applied Biosystems, Foster City, Calif.).

### Temperature Sensitivity Of The Mth RIR 1 Intein

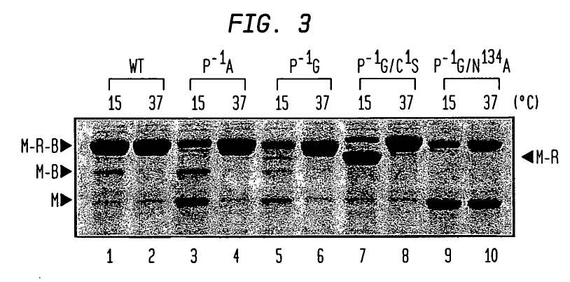

The cleavage and/or splicing activity of the Mth RIR1 intein was more proficient when protein synthesis was induced at 15° C. than when the induction temperature was raised to 37° C. (FIG. 3). The effect temperature has on the Mth RIR1 represents a way to control the activity of this intein for use in controlled splicing or cleavage reactions. Replacement of Pro⁻¹ with a Gly and Cys¹ with a Ser resulted in a double mutant, the pMRB9GS construct, which showed only in vivo C-terminal cleavage activity when protein synthesis was induced at 15° C. but not at 37° C. Another double mutant, the pMRB9GA construct, displayed slow cleavage, even at 15° C., which allowed the accumulation of substantial amounts of the precursor protein and showed potential for use as a C-terminal cleavage construct for protein purification.

1. A method for generating a specified amino acid at the N-terminus of a target protein, comprising:
expressing in a host cell, a nucleic acid encoding a fusion protein comprising an intein or modification thereof and a target protein wherein the intein-encoding sequence is 5′-proximal to a codon specifying the specified amino acid at the amino terminus of the target protein; and
cleaving the intein from the target protein so as to generate the specified amino acid at the N-terminus of the target protein.

2. The method of claim 1, wherein the nucleic acid sequence encoding the fusion protein further comprises a sequence encoding a protein-binding domain.

3. The method of claim 2, wherein the protein-binding domain is the chitin-binding domain.

4. The method of claim 3, wherein the fusion protein is purified on a chitin column.

5. The method of claim 1, wherein the nucleic acid is incorporated in a plasmid and the plasmid is capable of expression in a host cell selected from the group consisting of a bacterial, a yeast, a plant, an insect and a mammalian host cell.


SEQUENCE LISTING (26 sequences)

SEQ ID NO: 1 - DNA, 99 bp, Artificial Sequence
  Description of Artificial Sequence Chemically Synthesized From Methanobacterium thermoautotrophicum.
tcgaggcaaccaacccctgcgtatccggtgacaccattgtaatgactagtggcggtccgcgcactgtggctgaactggagggcaaaccgttcaccgcac

SEQ ID NO: 2 - DNA, 93 bp, Artificial Sequence
  Description of Artificial Sequence Chemically Synthesized From Methanobacterium thermoautotrophicum.
ccggttggctgctcgccacagttgtgtacaatgaagccattagcagtgaatgcgctagcaccgtaaacagtagcgtcataaacatcctggcgg

SEQ ID NO: 3 - DNA, 100 bp, Artificial Sequence
  Description of Artificial Sequence Chemically Synthesized From Methanobacterium thermoautotrophicum.
tgattcgcggctctggctacccatgcccctcaggtttcttccgcacctgtgaacgtgacgtatatgatctgcgtacacgtgagggtcattgcttacgttt

SEQ ID NO: 4 - DNA, 100 bp, Artificial Sequence
  Description of Artificial Sequence Chemically Synthesized From Methanobacterium thermoautotrophicum.
gacccatgatcaccgtgttctggtgatggatggtggcctggaatggcgtgccgcgggtgaactggaacgcggcgaccgcctggtgatggatgatgcagct

SEQ ID NO: 5 - DNA, 87 bp, Artificial Sequence
  Description of Artificial Sequence Chemically Synthesized From Methanobacterium thermoautotrophicum.
ggcgagtttccggcactggcaaccttccgtggcctgcgtggcgctggccgccaggatgtttatgacgctactgtttacggtgctagc

SEQ ID NO: 6 - DNA, 49 bp, Artificial Sequence
  Description of Artificial Sequence Chemically Synthesized From Methanobacterium thermoautotrophicum.
gcattcactgctaatggcttcattgtacacaactgtggcgagcagccaa

SEQ ID NO: 7 - DNA, 100 bp, Artificial Sequence
  Description of Artificial Sequence Chemically Synthesized From Methanobacterium thermoautotrophicum.
ccagcgccacgcaggccacggaaggttgccagtgccggaaactcgccagctgcatcatccatcaccaggcggtcgccgcgttccagttcacccgcggcac

SEQ ID NO: 8 - DNA, 90 bp, Artificial Sequence
  Description of Artificial Sequence Chemically Synthesized From Methanobacterium thermoautotrophicum.
gccattccaggccaccatccatcaccagaacacggtgatcatgggtcaaacgtaagcaatgaccctcacgtgtacgcagatcatatacgt

SEQ ID NO: 9 - DNA, 97 bp, Artificial Sequence
  Description of Artificial Sequence Chemically Synthesized From Methanobacterium thermoautotrophicum.
cacgttcacaggtgcggaagaaacctgaggggcatgggtagccagagccgcgaatcagtgcggtgaacggtttgccctccagttcagccacagtgcg

SEQ ID NO: 10 - DNA, 55 bp, Artificial Sequence
  Description of Artificial Sequence Chemically Synthesized From Methanobacterium thermoautotrophicum.
cggaccgccactagtcattacaatggtgtcaccggatacgcaggggttggttgcc

SEQ ID NO: 11 - DNA, 45 bp, Artificial Sequence
  Description of Artificial Sequence Chemically Synthesized From Methanobacterium thermoautotrophicum.
tcgaggcaaccaacgcatgcgtatccggtgacaccattgtaatga

SEQ ID NO: 12 - DNA, 45 bp, Artificial Sequence
  Description of Artificial Sequence Chemically Synthesized From Methanobacterium thermoautotrophicum.
ctagtcattacaatggtgtcaccggatacgcatgcgttggttgcc

SEQ ID NO: 13 - DNA, 36 bp, Artificial Sequence
  Description of Artificial Sequence Chemically Synthesized From Methanobacterium thermoautotrophicum.
tcgagggctgcgtatccggtgacaccattgtaatga

SEQ ID NO: 14 - DNA, 36 bp, Artificial Sequence
  Description of Artificial Sequence Chemically Synthesized From Methanobacterium thermoautotrophicum.
ctagtcattacaatggtgtcaccggatacgcagccc

SEQ ID NO: 15 - DNA, 54 bp, Artificial Sequence
  Description of Artificial Sequence Chemically Synthesized From Methanobacterium thermoautotrophicum.
tcgagggcatcgaggcaaccaacggatccgtatccggtgacaccattgtaatga

SEQ ID NO: 16 - DNA, 54 bp, Artificial Sequence
  Description of Artificial Sequence Chemically Synthesized From Methanobacterium thermoautotrophicum.
ctagtcattacaatggtgtcaccggatacggatccgttggttgcctcgatgccc

SEQ ID NO: 17 - DNA, 54 bp, Artificial Sequence
  Description of Artificial Sequence Chemically Synthesized From Methanobacterium thermoautotrophicum.
tcgagggcatcgaggcaaccaacggcgccgtatccggtgacaccattgtaatga

SEQ ID NO: 18 - DNA, 54 bp, Artificial Sequence
  Description of Artificial Sequence Chemically Synthesized From Methanobacterium thermoautotrophicum.
ctagtcattacaatggtgtcaccggatacggcgccgttggttgcctcgatgccc

SEQ ID NO: 19 - DNA, 28 bp, Artificial Sequence
  Description of Artificial Sequence Chemically Synthesized From Methanobacterium thermoautotrophicum.
gtacacgcatgcggcgagcagcccggga

SEQ ID NO: 20 - DNA, 28 bp, Artificial Sequence
  Description of Artificial Sequence Chemically Synthesized From Methanobacterium thermoautotrophicum.
ccggtcccgggctgctcgccgcatgcgt

SEQ ID NO: 21 - protein, 14 aa, Artificial Sequence
  Description of Artificial Sequence Chemically Synthesized From Methanobacterium thermoautotrophicum.
TLEGCGEQPTGXLK

SEQ ID NO: 22 - protein, 7 aa, Artificial Sequence
  Description of Artificial Sequence Chemically Synthesized From Methanobacterium thermoautotrophicum.
CGEQPTG

SEQ ID NO: 23 - DNA, 462 bp, Artificial Sequence
  CDS (1)..(462): Description of Artificial Sequence Chemically Synthesized From Methanobacterium thermoautotrophicum.
caactcgggaggatagaggcaaccaacccctgtgtatccggtgacaccattgtaatgacatccgggggtccgcggacagtggctgaactggagggcaagcccttcaccgcacttatcaggggctcagggtacccctgcccctcaggtttcttcaggacctgtgaacgggacgtatatgatcttagaaccagggagggtcattgcttaaggttgacccatgatcacagggtccttgtaatggatggtggtctggaatggcgtgccgccggtgaacttgaaaggggagaccgccttgtgatggatgatgctgcaggggagtttccggcacttgcaaccttcagaggcctcaggggcgccggccgccaggatgtctatgacgccactgtctacggtgccagtgcattcacagccaatggattcatagtccacaactgtggggagcagccactcctcacccatgaa

SEQ ID NO: 24 - protein, 154 aa, Artificial Sequence
  Description of Artificial Sequence Chemically Synthesized From Methanobacterium thermoautotrophicum.
QLGRIEATNPCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHNCGEQPLLTHE

SEQ ID NO: 25 - DNA, 447 bp, Artificial Sequence
  CDS (1)..(447): Description of Artificial Sequence Chemically Synthesized From Methanobacterium thermoautotrophicum.
ctcgaggcaaccaacccctgcgtatccggtgacaccattgtaatgactagtggcggtccgcgcactgtggctgaactggagggcaaaccgttcaccgcactgattcgcggctctggctacccatgcccctcaggtttcttccgcacctgtgaacgtgacgtatatgatctgcgtacacgtgagggtcattgcttacgtttgacccatgatcaccgtgttctggtgatggatggtggcctggaatggcgtgccgcgggtgaactggaacgcggcgaccgcctggtgatggatgatgcagctggcgagtttccggcactggcaaccttccgtggcctgcgtggcgctggccgccaggatgtttatgacgctactgtttacggtgctagcgcattcactgctaatggcttcattgtacacaactgtggcgagcagccaaccggtgaattc

SEQ ID NO: 26 - protein, 149 aa, Artificial Sequence
  Description of Artificial Sequence Chemically Synthesized From Methanobacterium thermoautotrophicum.
LEATNPCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHNCGEQPTGEF